(19) United States
(12) Patent Application Publication
(10) Pub. No.: US 2004/0147437 A1
(43) Pub. Date: Jul. 29, 2004

(54) Calycins

(76) Inventor: John Findlay, Leeds (GB)

Correspondence Address: MYERS BIGEL SIBLEY & SAJOVEC, PO BOX 37428, RALEIGH, NC, 27627, US

(21) Appl. No.: 10/362,714

(22) PCT Filed: Aug. 24, 2001
(86) PCT No.: PCT/GB01/03813

(30) Foreign Application Priority Data
Aug. 24, 2000 (GB) 0020749.8

(51) Int. Cl. A61K 38/17; G01N 33/53

(52) U.S. Cl. 514/12; 435/7.1

(57) ABSTRACT

The invention relates to the use of calycins, and in particular, the use of lipocalins in the transport and/or binding of ligands to a substrate wherein said substrate is not hair or skin: and also the modification of calycins to alter the specificity and/or affinity of said calycins for said substrate and/or ligands.

The invention relates to the use of calycins, and in particular, the use of lipocalins in the transport and/or binding of ligands to a substrate wherein said substrate is not hair or skin; and also the modification of calycins to alter the specificity and/or affinity of said calycins for said substrate and/or ligands.

Lipocalins are a diverse family of extracellular proteins found in biological organisms. They display various functions related to the binding and transport of ligands. For example, they are involved in mediating pheromone activity, olfaction, taste, vision, immunomodulation and general functions relating to cellular homeostasis.

Arguably the most extensively studied lipocalin is the retinol binding protein (RBP) which transports retinol around the body. Retinol (or vitamin A) is an extremely important substance which is crucial to the normal functioning of the visual, the reproductive and the immune systems, and in haematopoiesis. Metabolites of retinol are also active in development, differentiation and against cancer cells.

The 3-dimensional structure of RBP was determined in 1984 (Newcomer, M. E., Jones, T. A., Aqvist, J., Sundelin, J., Eriksson, U., Rask, L. and Peterson, P. A. (1984) EMBO J. 3 1451-1454) and revealed a novel structure. As many more lipocalins were identified and characterised, it became apparent that this basic eight-β-stranded structure was conserved in all the structural homologues although the overall level of amino acid sequence identity is low. This family of proteins became known as the lipocalins (derived from the Greek word lipos, meaning fat and calyx meaning cup). A closely related family of 10-β-stranded intracellular protein has also been found, the two groups together comprising the calycins.

### CONFIRMATION COPY

The lipocalins are found throughout biological life and range in molecular weight from approximately 18 kDa to 45 kDa. However, this represents the monomeric molecular form; many lipocalins exist in as multimers. For example the bilin-binding protein exists as a homotetramer of a 19.5 kDa monomeric subunit. Apolipoprotein D exists as a dimer, but is also associated with other proteins. RBP is found complexed with TTR in up to a 1:1 ratio, while crustacyanin appears to contain 16 subunits in its stable molecular form.

As mentioned previously, the lipocalins are not highly conserved at the amino acid level, but do retain certain structural features that make them recognisable as lipocalins. The core structure is represented by orthogonally arranged β-sheets, the β-strands connected to each other to form a barrel-like structure closed at one end, thereby producing the ‘cup shaped’ structure. The cavity thus created represents the binding pocket for the ligand transported/bound by the lipocalin. There are some short regions of sequence homology that help to identify a protein as a lipocalin. The two most definitive but not obligatory being the GXW and TDY motifs.

The major function of the lipocalins is the binding and transport of specific ligands (although at least one has enzymic activity) which are usually small hydrophobic molecules. The specificity of binding is determined by the conformation and constituent side-chains of the lipocalin pocket. It is of note that in vitro many lipocalins can bind with high affinity to a range of hydrophobic molecules not normally encountered in nature. This may represent an inherent ability of the lipocalins to bind molecules having particular biochemical and structural properties.

Another example of a lipocalin which has been extensively studied is β-lactoglobulin. β-lactoglobulin is a very abundant protein found in the milk of mammals. The monomer molecular weight of bovine β-lactoglobulin is 18 kDa, corresponding to 162 amino acids. A number of investigators have shown that β-lactoglobulin binds retinol and fatty acids in vitro. However, the exact role played by β-lactoglobulin in vivo is still not understood.

In simple terms a calycin can be functionally divided into a “binding domain” and a “targeting domain”. The “binding domain” functions to interact with ligands and the “targeting domain” functions to provide specificity in transporting the bound ligand to a defined site.

In some instances the binding domain may also be part of the targeting mechanism.

In our co-pending application, PCT/GB00/00517, we discussed the potential of β-lactoglobulin to bind fatty acids at a non-native site. The term non-native is defined as a targeting site not naturally encountered by a lipocalin. It will be apparent to the skilled artisan that said binding domain and/or said targeting domain may be endogenous to said calycin. However, in the instance where said calycin would not naturally bind said agent, the corresponding binding and/or targeting domain is adapted accordingly. This adaption may comprise either the alteration of the existing binding and/or targeting domain or the substitution of same for a domain that has the required functionality.

The hair cuticle is thought to be coated in fatty acids, these fatty acids may function as a hydrophobic barrier to water and they also give hair its natural sheen and texture. Cosmetic hair conditioners function to accentuate these features of hair. However, conditioners currently available only have a transient association with the hair cuticle and therefore the user has to periodically apply conditioner to maintain the sheen and body of the hair. Our co-pending application PCT/GBOO/00517 demonstrates that lipocalins, for example β-lactoglobulin, mouse urinary proteins (MUP) and equivalents from other species can be adapted to provide a conditioning property to hair.

Additionally or alternatively multimeric complexes of lipocalins can be adapted to carry more than one agent. For example, and not by way of limitation, multimeric lipocalin complexes can be used to carry both conditioning agents and fragrances to hair. Fragrance molecules are generally volatile. The binding of fragrance molecules to a lipocalin provides for delayed and/or controlled release. One example of such a lipocalin is the pyrazine-binding protein which binds bell-pepper odourant 3-isobutyl-3-methoxypyrazine. Other examples are MUP and equivalents.

Alternatively lipocalins can be selected from libraries to bind an odourant of choice. Huge libraries of randomly mutated calycins generated by recombinant DNA technologies are designed to introduce mutations into selected genes. From these libraries particular binding specificities for specific ligands (eg fragrances, drugs, antibiotics, pigments) are detected and isolated.

The pigmented lipocalin, crustacyanin, has been sequenced and modelled by us. The ligand in this instance is a carotenoid, astaxanthin, and there are a number of such lipocalin-carotenoid complexes in nature. The interaction between carotenoid and lipocalin produces a change in the absorbance characteristics of the astaxanthin such that the complex now assumes a different colour. This system provides a colour-based sensor which can absorb at different wavelengths and be sensitive to changing environmental conditions such as temperature, time, pH, microbial culture, addition or removal of agents. Mutants, generated as above, can have defined sensitivities to ligands and environmental conditions.

Statements of the Invention

According to the invention there is provided a modified calycin monomer comprising:

i) a binding domain adapted to bind a ligand; and

ii) a targeting domain adapted to bind a substrate characterised in that the modified calycin monomer is used to target a ligand to a substrate to which it would not naturally bind.

The ligand binding domain binds a ligand which typically, but by no means exclusively, comprises a hydrophobic molecule. The ligand may be one that is not normally encountered in nature.

The substrate to which the calycin is located may include any substrate excluding that of hair or skin. By way of example, the substrate to which the calycin is located may comprise clothes fibres, particularly where the calycin is a fragrance binding calycin and is used in washing powders and other such laundry products. Where the calycin is used in the binding and delivery of therapeutic molecules, the substrate to which the calycin is located may comprise the surface of a cell.

Other potential substrates include surfaces such as provided by paper (cellulose), chitin, collagen, glass, metal, synthetic polymers and so on, all of which possess moieties which can be bound by native or mutated lipocalins or by specific binding proteins/domains attached to the lipocalin.

An engineered calycin monomer can be made up of two native monomers fused chemically or by molecular biology methods thereby generating two, or more specificities or a classically monomeric form can bind one entity/moiety on an otherwise polymeric substrate. In a preferred embodiment of the invention said modified calycin monomer is adapted to bind at least two ligands. Preferably said ligands are the same. Alternatively said modified calycin monomer is adapted to bind at least two different ligands.

In a further preferred embodiment of the invention said modified calycin monomer is a multimeric complex. Preferably said multimersised complex comprises identical calycin monomers. Alternatively said complex comprises different calycin monomers.

The calycin complex can comprise monomers which bind different ligands but contain the same targeting domain to facilitate the targeting of different ligands to the same surface. Alternatively the complex can comprise monomers which bind the same ligand but contain different targeting domains to facilitate the targeting of the same ligand to different surfaces.

In a further preferred embodiment of the invention said modified calycin monomer further comprises an interaction domain which facilitates the multimerisation of calycin monomers into a complex. Examples include bovine OBP and members of the crustacyanin-like group which have additional elements which participate in subunit: subunit interactions.

Preferably the multimerisation of the calycin monomers into a complex is achieved via peptide linkage. Accordingly, the nature of the interaction domain is preferably such that the formation of such a linkage between at least two monomers is facilitated. The interaction domain may be a naturally occurring part of the calycin or may be inserted using synthetic or recombinant techniques, for example conventional techniques.

In an alternative preferred embodiment of the invention said modified calycin monomers are multimerised by crosslinking agents. Preferably said crosslinking agent is a bifunctional protein cross-linking agent. Cross-linking agents may be homo-bifunctional or hetero-bifunctional.

Cross linking agents such as DSS, DMA, EDC are well known in the art and are used to cross-link proteins to one another via functional groups. Typically said cross-linking agents link proteins through free amino groups or between sulphydryl groups in sulphur containing amino acids such as cysteine. Crosslinking agents may produce either covalent linkages or non-covalent linkages which can be broken by changing the milieu surrounding the cross-linked protein complex (eg addition of a reducing agent).

According to a further aspect of the invention there is provided a method to multimerise modified calycin monomers comprising:

i) providing modified calycin monomers with conditions and crosslinking reagents sufficient to cross-link monomers into multimeric complexes; and optionally

ii) purifying the cross-linked complexes from the reagent mix.

In a further aspect of the invention there is provided a modified calycin monomer which is altered by deletion, substitution or addition of at least one amino acid residue wherein said alteration alters the specificity of the binding domain for at least one ligand. Alternatively, or in addition, said alteration alters the affinity of said binding domain for at least one ligand.

FIG. 4 represents the protein maps of natural isolated proteins rERAPB and rMUP. These proteins were used as a template to construct several recombinant proteins as shown.

Mutation of Arg 80 and Glu63 to isoleucine in Epididymal Retinoic acid Binding Protein (ERABP) reduced affinity for retinoic acid and caused the mutant protein to bind retinol and retinal. The native protein is incapable of binding these latter two ligands (see FIG. 5).

In a yet further aspect of the invention there is provided a modified calycin monomer which is altered by deletion, substitution or addition of at least one amino acid residue wherein said alteration either alters the specificity of the targeting domain of said modified calycin monomer for a selected surface. Alternatively, or in addition, said alteration alters the affinity of said targeting domain for a selected surface.

In a preferred embodiment of the invention said altered modified calycin monomer is multimerised into a complex according to any previous embodiment of the invention. Methods for creating the above described altered calycins are well known in the art and comprise recombinant DNA techniques in the creation of calycin monomers and fusion proteins. It will also be apparent to those skilled in the art that the affinity of a calycin for an agent can be altered, for example, by genetically modifying the binding domain, to create an adapted calycin that has a higher affinity for said agent. Moreover, the binding domain may be genetically modified in this way to alter the specificity of agent binding. It is also apparent that specificity of the targeting domain can be altered to either alter the specificity of targeting, or alternatively, increase or decrease the affinity of the targeting domain for its binding site on a selected surface. Genetic modification of this type is well known in the art and include, for example, the introduction of point mutations to alter the properties of the agent binding site and/or the targeting domain.

It is also apparent that the production of molecular complexes with more than one type of agent binding domain can be produced. This can be achieved by the fusion of genes for the calycins to one another, with appropriate linking regions to produce a multi component gene and gene product. Or, interaction sites can be introduced into individual monomers of the calycins such that on mixing the individual proteins, molecules assemble into multi-sub unit complexes with similar or different functionalities. Genetic modifications of this type are well known in the art and include the introduction of point mutations, additions, deletions etc. to alter the properties of the protein.

According to a further aspect of the invention there is provided a vector which includes nucleic acid which encodes a modified calycin monomer according to any previous aspect or embodiment of the invention. By way of example and by no means of limitation, pQE-30 vectors may be used for expression in E. Coli strains (eg SG13009) containing native or mutated calycin sequences. The pPIC3.5 vector may be used for expression in yeast strains e.g., Pichia pastoris strains eg MSD1168-his 4 or GS115-his 4 containing native or mutated calycin sequences. These are all commercial vectors/strains.

Preferably said vector is adapted for the recombinant production of said modified calycin monomer.

Typically said adaptation includes, by example and not by way of limitation, the provision of transcription control sequences (promoter sequences) which mediate cell/tissue specific expression. These promoter sequences may be inducible, repressible or constitutive. Adaptations also include the provision of selectable marker and autonomous replication sequences which both facilitate the maintenance of said vector in either the eukaryotic cell or prokaryotic host. Adaptations which facilitate the expression of vector encoded genes include the provision of transcription termination/polyadenylation sequences. This also includes the provision of internal ribosome entry sites (IRES) which function to maximise expression of vector encoded genes arranged in bicistronic or multi-cistronic expression cassettes.

These adaptations are well known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc. (1994).

Particular site-specific mutants are made by use of a commercially available methodology called, QuikChange™ Site-Directed Mutagenesis, available from Stratagene, La Jolla, Calif., USA.

Mutant lipocalin libraries are generated by the published methodology known as “DNA Shuffling”: STEMMER, W. P. C. (1994) Nature 370 389-391. STEMMER, W. P. C. (1994) Proc. Natl. Acad. Sci. USA 91 10747-10751

For the purpose of targeting particular mutant lipocalins to a specific receptor, the three loop regions of the lipocalin molecule (responsible for targeting) are changed using a PCR-based methodology employing oligonucleotide primer pairs that contain, between them, the sequence for the loop region to be inserted. In such reactions the sequence of the loop to be removed is lost and replaced by the sequence encoded in the oligonucleotide primers.

According to a yet further aspect of the invention there is provided a cell or cell-line transformed or transfected with the vector according to the invention. By way of example and by no means of limitation E coli strains including SG13009 and yeast strains including Pichia pastoris may be used.

In yet a further aspect of the invention there is provided a method to recombinantly manufacture modified calycin monomers according to the invention comprising:

i) growing said cell or cell-line transformed or transfected with the vector according to the invention in conditions conducive to the manufacture of said polypeptide; and

ii) purifying said modified calycin monomer from said cell, or its growth environment.

In a preferred method of the invention said vector encodes, and thus said modified calycin monomer is provided with, a secretion signal or affinity tag (e.g., His₆ or the streptavidin peptide) to facilitate purification of said monomer.

In accordance with any previous aspect or embodiment of the invention said modified calycin monomer or multimer is adapted for use in the targeting of at least one agent to the surface fibre of a laundry item.

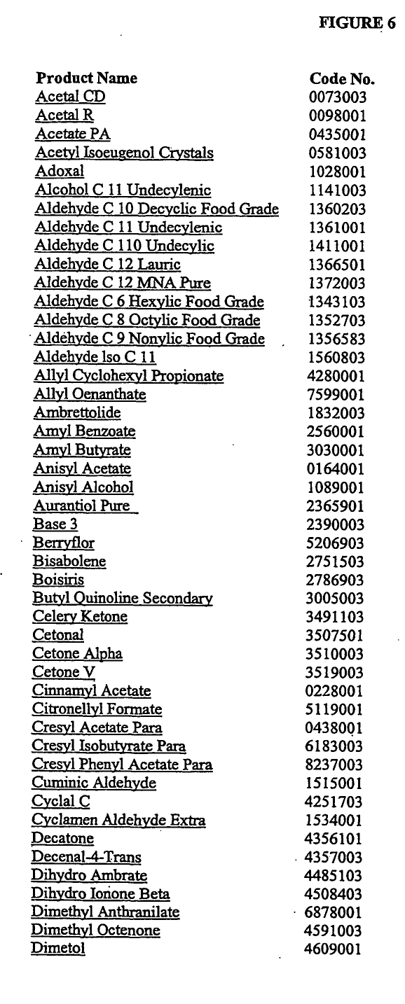

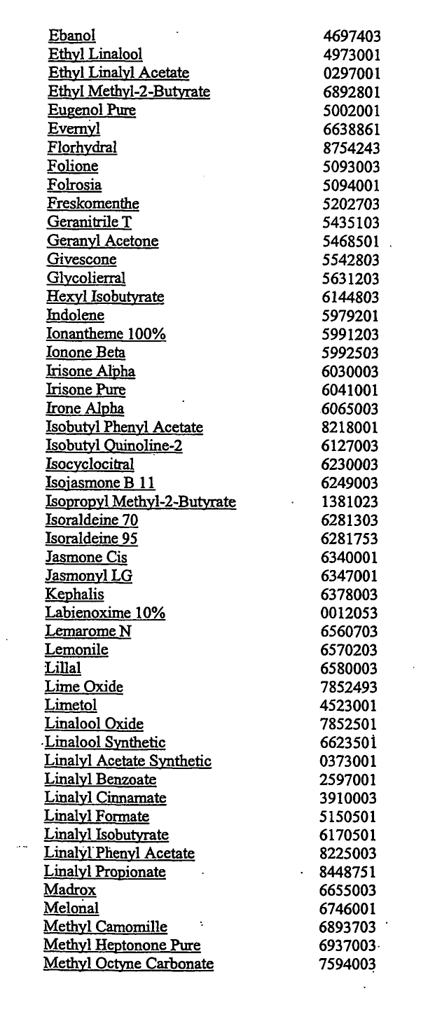

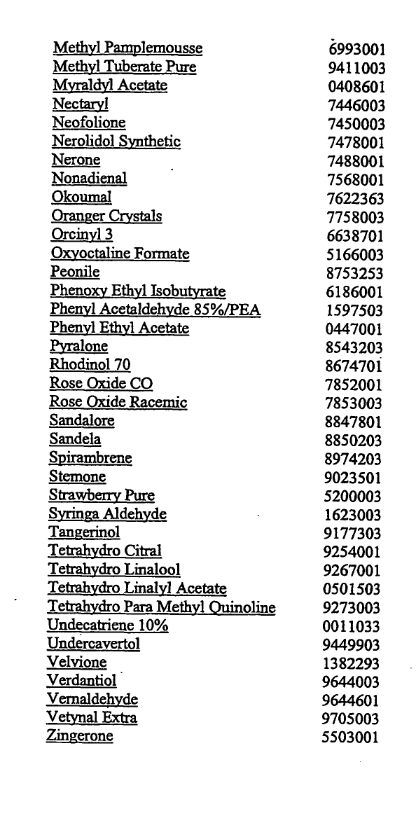

In a preferred embodiment of the invention said agent comprises a fragrance which confers a desired smell on said laundry item. Fragrance molecules of use are listed in FIG. 6 and include, by way of example only, β-ionone, jasmone, damascone and related molecules.

In a further preferred embodiment said agent comprises a conditioner, e.g., silicone, which confers a pleasant texture to said laundry item.

In a yet further preferred embodiment said agent comprises a protective agent, e.g., insecticide, insect repellant or UV protectant, which confer a protective property to said laundry item.

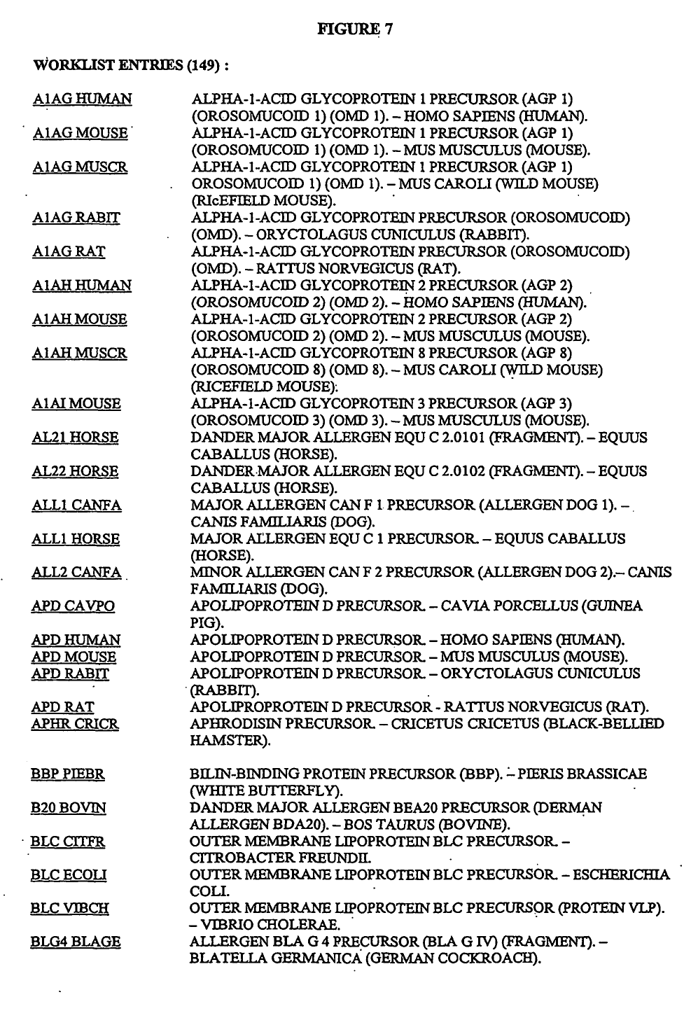

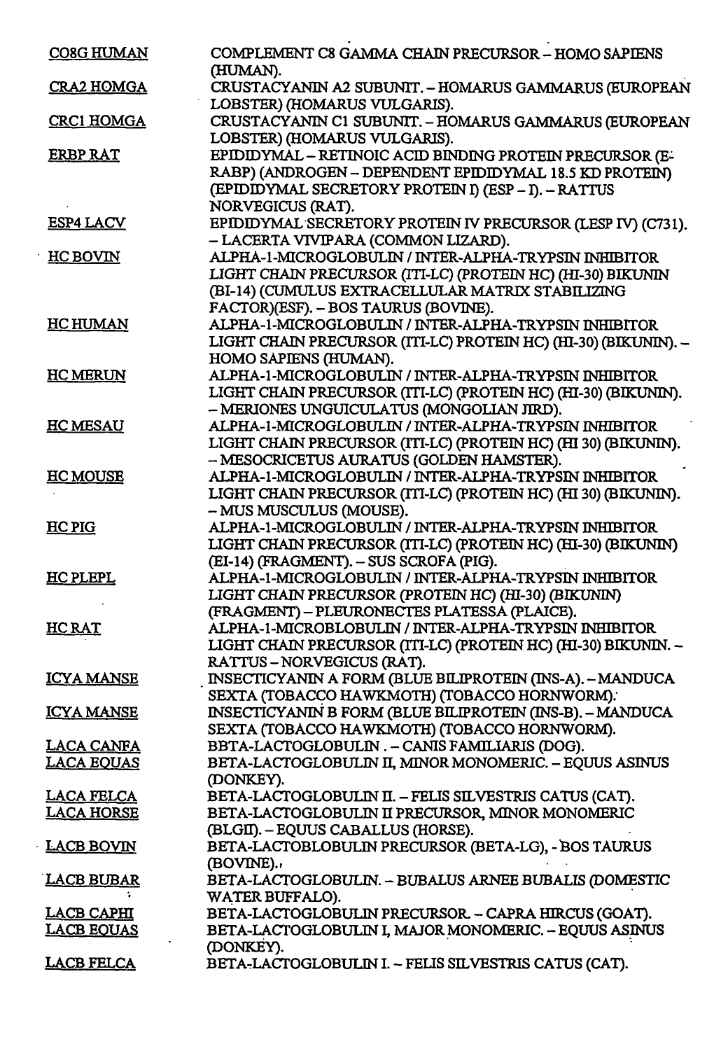

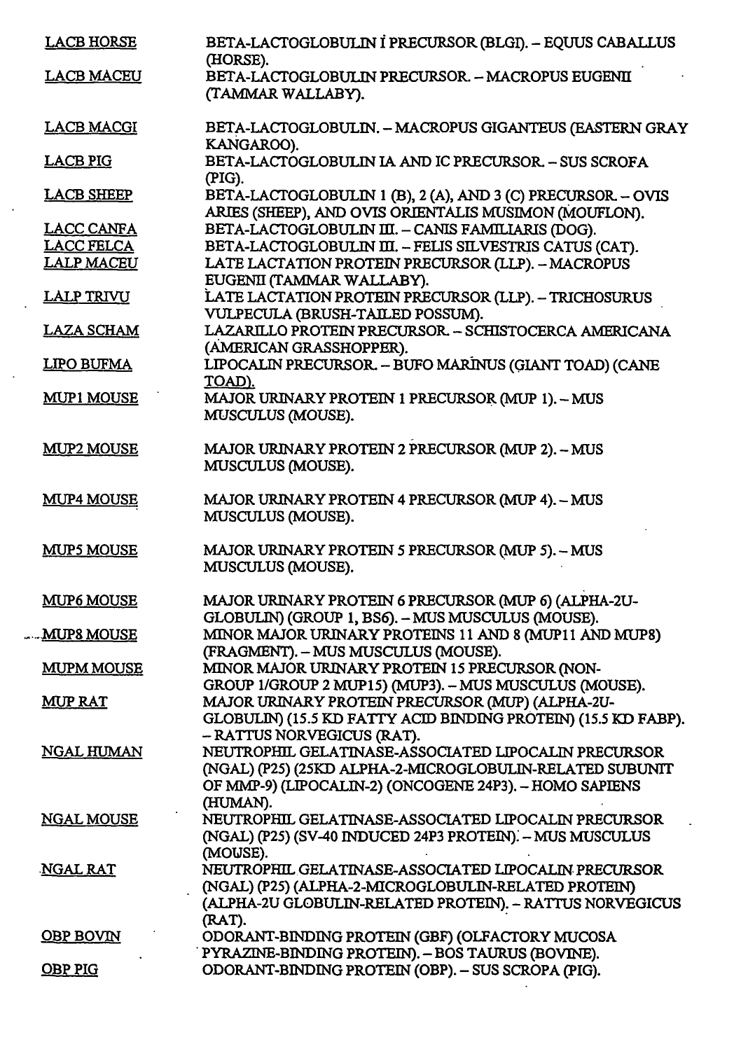

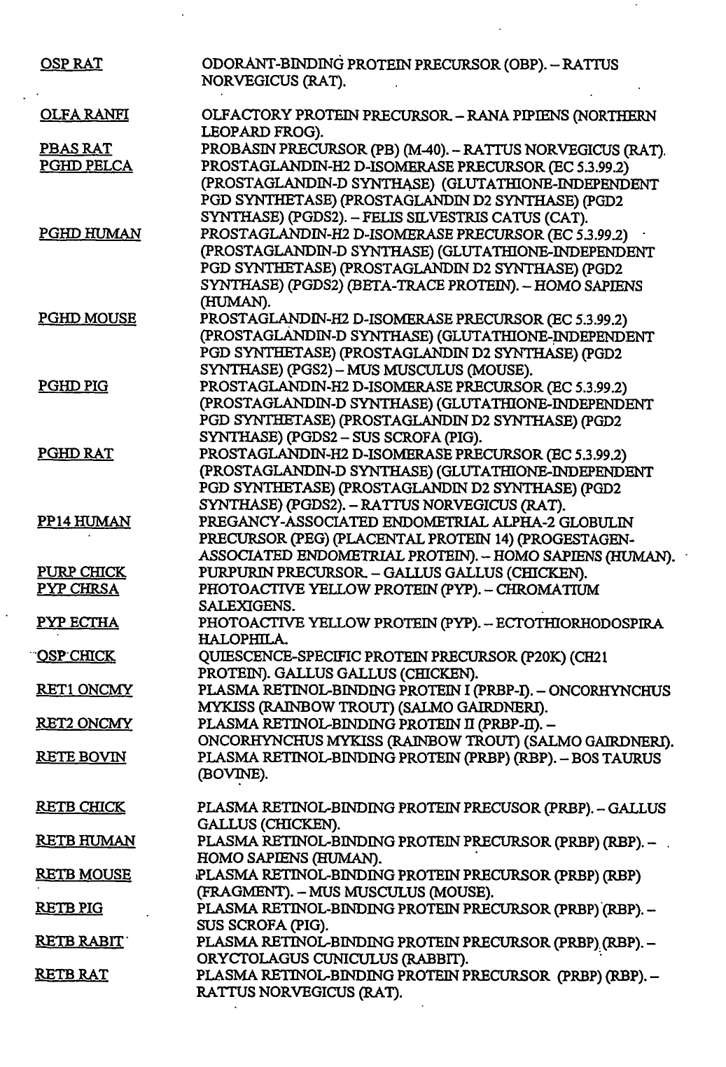

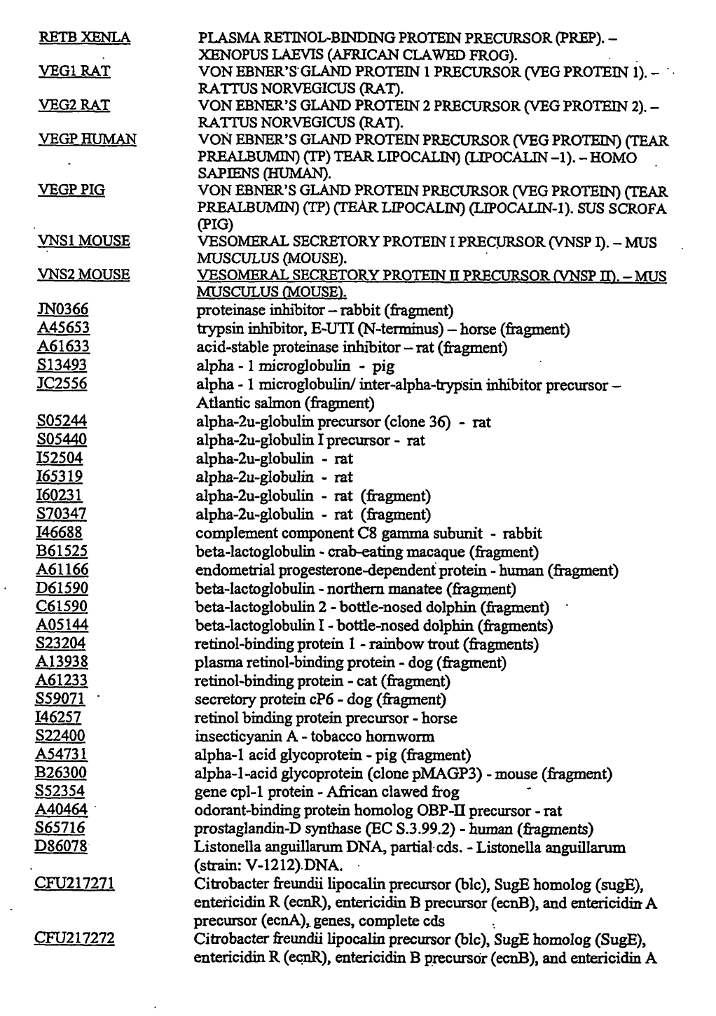

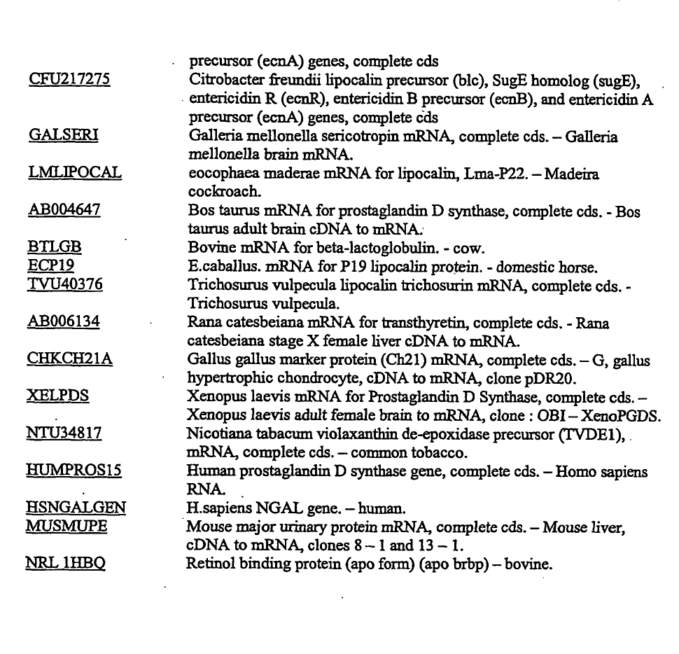

In a further preferred embodiment of the invention said calycin is selected from any of those listed in FIG. 7 and is most preferably selected from any of the following; Mouse Urinary Protein (MUP) and equivalents thereof, rat alpha 2 microglobulin odour binding proteins (OBP), other odour-binding proteins and equivalents thereof, β-lactoglobulin, retinal and retinoid binding proteins, alpha 1 microglobulin, fatty acid binding proteins, intracellular retinoid binding proteins— all involved in hydrophobic ligand binding and which can be modified to include other specificities eg antibiotics, drugs, pigments and so on. Crustacyanin, insectacyanin are preferred examples of pigmented lipocalins.

Any of these can be a monomer and participate in multimer formation using chemical or molecular biology methodology.

In accordance with any previous aspect or embodiment of the invention, said modified calycin monomer or multimer is for use as a biosensor. Of particular importance here are the pigmented lipocalins (native or mutated versions) which are, by way of example, heat, light or gas sensitive and whose response involves a colour change. Mutants, generated as above, can also have defined sensitivities to ligands and environmental conditions.

In accordance with any previous aspect or embodiment of the invention, said modified calycin monomer or multimer is for use in the targeting of at least one therapeutic agent to at least one surface, e.g., a cell surface. The therapeutic agent can then be transferred into the cell, thereby effecting drug targeting, delivery and uptake. The nature of the therapeutic agent can vary considerably. Examples include anti-cancer drugs, antibiotics and channel modifiers.

Specific examples of targeting include;

(i) targeting to a cell surface via receptor recognition sequences, thereby facilitating uptake via that surface of therapeutic agents such as anti-cancer drugs, antibiotics, neurological drugs, receptor and ion channel modulators,

(ii) attachment to a surface such as a patch or wound dressing to effect slow delivery of biologically active agents eg vitamins, growth factors, antibiotics and signal molecules.

From the libraries of mutants of lipocalins, e.g., MUPs, proteins can be isolated which have particular specificities for defined therapeutic agents. The lipocalin is then subject to further mutagenesis such that residues 30-41, 59-69 and 87-101 on RBP, or equivalent to these on MUP, are inserted into the lipocalin isolated from said libraries. These reconstructed lipocalins are then utilised for delivery of the ligand they carry to biological membranes containing the appropriate receptor.

Accordingly, the invention includes alteration of regions of the calycin (i) to affect binding of the calycin/lipocalin to its cell membrane receptor and other carriers or (ii) by production of chimeras e.g., of ERABP and RBP and MUP to redirect the lipocalcin core to a different receptor.

A preferred embodiment of the invention involves replacement of residues 21-37, 54-59 and 77-84 in ERABP by residues 30-41, 59-69 and 87-101 inclusive respectively of RBP, causing the ERABP mutant to now interact in a ligand-dependent fashion to transthyretin and to the RBP receptor.

Similarly, replacement of residues 58-63 and 82-87 in MUP by 59-68 and 87-101 inclusive respectively of RBP cause the MUP/RBP chimera to interact in a ligand-dependent fashion with transthyretin and with the RBP receptor. These regions are examples of specific targeting which can be transferred from lipocalin to lipocalin.

An embodiment of the invention will now be described, by example only, with reference to the following figures, methods and materials wherein;

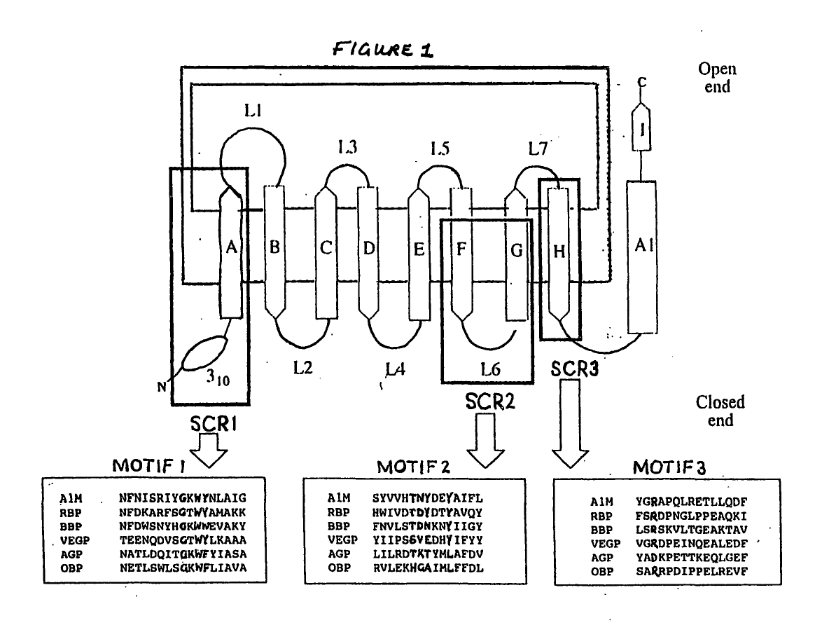

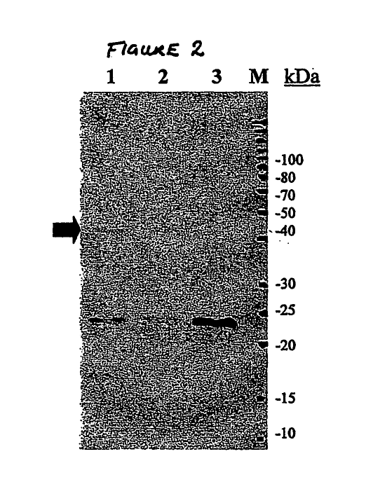

FIG. 1 is a diagrammatic representation of conserved characteristics found in many calycins.

FIG. 2 represents an autoradiograph showing further evidence of EDC-induced β-LG/rMUP heterodimer formation wherein;

Lane 1 represents [³⁵S]Met-labelled rMUP+β-LG with 0.5 mM EDC

Lane 2 represents [³⁵S]Met-labelled rMUP with 0.5 mM EDC

Lane 3 represents [³⁵S]Met-labelled rMUP, untreated

M indicates a gel lane containing molecular mass markers.

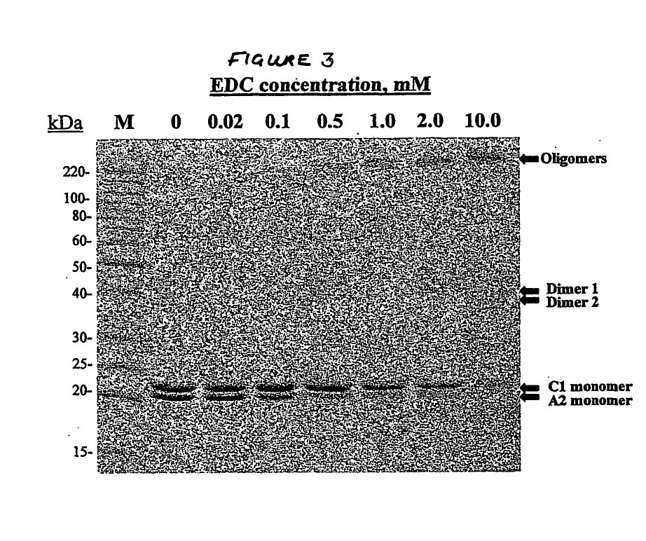

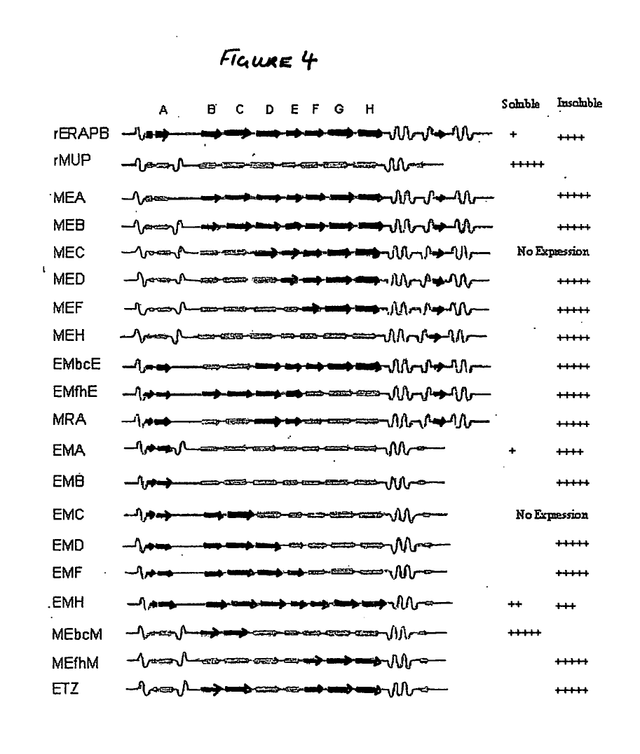

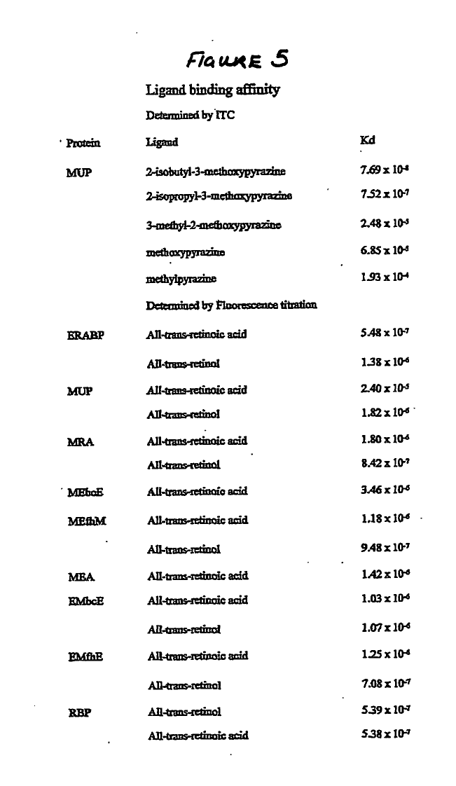

FIG. 3 depicts a protein gel showing EDC-induced oligomerisation of α-Crustacyanin wherein M indicates a gel lane containing molecular mass markers.

FIG. 4 represents the protein maps of the natural isolated proteins rERAPB and rMUP and several recombinant proteins.

FIG. 5 shows the ligand binding affinity of various proteins for selected ligands.

FIG. 6 provides an exemplary list of fragrance molecules which may be employed in an embodiment of the invention.

FIG. 7 provides an exemplary list of calycins which may be employed in an embodiment of the invention.

Materials and Methods

Methods for Generation of Calycin Multimers

There are a number of possible approaches to generating multimers of calycin molecules. Multimers generated can be homomeric or heteromeric and may comprise two calycin molecules (dimers) or higher order complexes (trimer and above). Preferred routes to generate heterodimers by chemical means are outlined below.

Heterodimers Cross-Linked Via the N-Termini

Disuccinimidyl-suberimidate-dihydrochloride (DSS).

DSS is an homobifunctional reagent (N-hydroxysuccinimide ester) which reacts with primary amine groups in proteins linking them via their N-terminal amine or surface-exposed lysine amino groups. DSS was prepared as a fresh stock solution of 20 mg/ml in ice-cold 25 mM Na₂HPO₄/1 mM-MgCl₂ (pH 8.0) and added to a 1 mg/ml calycin preparation in the same buffer to give a working concentration of 0.5, 2.0 and 10 mg/ml. The reactions were allowed to proceed at room temperature (21° C.) for 1 hour and quenched by the addition of 50 μL of 1.0M-ammonium acetate per ml of reaction mixture.

Dimethyl-adipimidate-dihydrochloride-(DMA)

Conditions were identical to DSS treatment.

1,5-difluoro-2,4-dinitrobenzene (DFDNB)

DFDNB was prepared as a 1.0M stock solution and added to the calycin suspension in 25 mM Na2HPO₄/1mM MgC12 (pH 8.0) to give a final concentration of 5.0 mM. The reaction was terminated after 30 minutes at room temperature by dialysis against a large volume of 0.1M ammonium acetate (pH7.0).

N-hydroxysuccinimidyl 2,3-dibromopropionate (SDBP)

SDBP is a heterobifunctional reagent (dibromopropionate and N-hydroxysuccinimide ester) used in sequential reactions to form the cross-link between the calycins. SDBP was prepared as a stock solution according to the manufacturer's instructions. The first calycin at a concentration of 1 mg/mlwas reacted with the N-hydroxysuccinimide moiety of SDBP added with constant stirring in phosphate buffer at pH 7 at 4° C. for 1 hr at an optimal concentration of SDBP. Excess cross-linker was removed by gel filtration and the derivatised calycin (now containing alkyldibromide groups on its surface) was reacted with the second calycin (total protein concentration of 1 mg/ml) by elevation of the temperature to 21° C. This reaction was also carried out with constant stirring under the same buffer and pH conditions as for the first reaction.

Heterodimers Cross-Linked Via Amino and Carboxyl Groups

1-Ethyl-3-[3-dimethylaminopropyl]carbodiimide hydrochloride (EDC)

1-Ethyl-3-[3-dimethylaminopropyl]carbodiimide hydrochloride (EDC) is used to catalyse the formation of an amide bond between the N-terminal amino group (or side chain amino group of lysine) of one calycin and the C-terminal carboxyl group of a second calycin to form the desired heterodimer. EDC was prepared as a stock solution according to the maunfacturer's instructions. Protein was suspended to a concentration of 1.0 mg/ml in 2-[N-morpholino]-ethanesulphonic acid (MES) buffered saline at pH 4.5-5.0 and EDC added with stirring for 16 hr at 25° C. at an optimal concentration of EDC.

Heterodimers Cross-Linked Via One Amino Group and One Free Cysteine Residue.

Succinimidyl 4-[N-maleimidomethyl]-cyclohexane-l-carboxylate (SMCC)

Succinimidyl 4-[N-maleimidomethyl]-cyclohexane-l-carboxylate (SMCC) is a heterobifunctional cross-linking agent (N-hydroxysuccinimide ester and maleimide) that is used to sequentially react with the N-terminal amino group (or side chain amino group of lysine) of one calycin and a free sulphydryl group on the other. SMCC was prepared as a stock solution according to the manufacturer's instructions. The reaction with the N-hydroxysuccinimide moiety was carried out in phosphate buffer at pH7.0 with constant stirring at 4° C. for 60 min at an optimal concentration of SMCC. Excess cross-linker was removed by gel filtration and the derivatised calycin (now with attached maleimide moiety) reacted with a surface-exposed cysteine side chain on the second calycin. The maleimide reaction was performed in phosphate buffer at pH 6.5-7.5 with constant stirring at 4° C. for 2.0 hr with a final protein concentration of 0.1-1.0 mg/ml. This reaction is preferred where the first calycin lacks a free cysteine and the second calycin contains one.

Mixtures of undesirable calycin homodimers and the desired calycin heterodimers are isolated by a combination of two affinity chromatography steps that recognise the binding properties of both calycins. For most of the cross-linking agents outlined above analogues are available with different spacer arm lengths that could be used as necessary.

In general, for most of the cross-linking reactions outlined above the cross-linking reagents are used in 2-50 fold molar excess over protein; the actual concentration of cross-linker employed would also depend upon the protein concentration used. After completion of a cross-linking reaction excess or hydrolysed cross-linker is quenched and removed by gel filtration, dialysis or centrifugal concentration according to well established practices in the art. The exact conditions of any particular cross-linking reaction, in terms of the reaction pH, temperature, time and concentration of protein and cross-linker, would need to be optimised for that particular reaction through experimentation.

Formation of Homodimers, Heterodimers and Oligomers From α-Crustacyanin

Native α-crustacyanin (˜0.3 mg/mL) isolated from lobster tissues was incubated with various concentrations of EDC ranging from 20 μM to 10 mM at pH 4.5 for 20 hr at room temperature. Samples were analysed by SDS-gel electrophoresis. The resulting Coomassie blue stained gel is shown in FIG. 3.

FIG. 3 shows that this calycin is very efficiently cross-linked to form very large oligomeric species which are likely to reflect the oligomeric nature of the native protein itself, i.e. proposed to be an octomer of non-covalently associated heterodimers comprised of monomers, C1 and A2.

FIG. 3 shows the two monomers clearly resolved in the untreated sample and as the concentration of the EDC cross-linker is increased, the A2 monomer is preferentially cross-linked followed by the C1 subunit into oligomers of increasing size. With 10 mM EDC virtually all the monomer is cross-linked and small amounts of dimers are visible.

The data presented here show that calycin monomers can be readily cross-linked into homodimers, heterodimers and higher oligomers. Such reactions will allow the formation of calycin species that possess two or more distinct and specific binding pockets. The degree to which homodimers, heterodimers and oligomers are formed is dependent on the specific calycin(s). Where the calycin monomers are initially in close non-covalent association with each other, as in the case of α-crustacyarin, very efficient cross-linking can be achieved.

Preparation of Ligand-Lipocalin Complexes

Typically, the lipocalin protein, in phosphate buffered saline, pH7.4 (PBS) is incubated with a 2-fold molar excess of ligand for 1 hour at 37°. Ligand-lipocalin complexes are separated from unbound ligand by gel filtration using Sephadex G-25 (20×1 cm column) equilibrated and developed with, for example, 2 mM Tris-HC1, pH9.0.

Measurement of Radiolabelled Lipocalin Binding to Cell Membranes and Cells

The binding of radiolabelled lipocalin to cell membranes and cells is measured using an oil centrifugation technique or by filtration and washing through filters. For oil centrifugation, target cell membranes (1-2 mg of protein/ml) or cells (1-2×10⁶ cells/ml) in PBS (plus ovalbumin) are incubated with radiolabelled lipocalin (2-10 nM) in a final volume of 100 μl. After incubation at the desired temperature for the desired time, samples are centrifuged at 12,500 g in a microcentrifuge for 2 minutes. Samples are then overlaid by an appropriate mixture of dibutyl phthalate and dinonyl phthalate (typically 3:2, v/v) and centrifuged again at 12,500 g for 2 minutes. The tubes are frozen in dry-ice and the tube bottoms, containing the cell or membrane pellets, cut off and measured for radioactivity. Non-specific binding of radiolabelled lipocalin was measured in the presence of at least 2 μM unlabelled lipocalin.

For the membrane filtration method, after the completion of the incubation of radiolabelled lipocalin with cell membranes or cells, samples are filtered, using suction, through glass fibre membrane filters and the filters similarly washed 3-4 times with several mls of PBS. Radioactivity on the filters is then determined.

Assay of Ligand Uptake From Lipocalin

Assay of ligand uptake from radiolabelled ligand-lipocalin complexes by membrane vesicles or cells was performed by an oil centrifugation method similar to that descried above except that in this case the ligand is radiolabelled. In some cases, where tritium-labelled ligands are used, the oil mixture is layered over a 50 μl aliquot of 5% (w/v) sucrose in PBS and the labelled test samples placed onto the oil layer prior to centrifugation. After freezing in dry-ice the tubes were cut at the oil/sucrose interface. The tube bottoms were incubated in 200 μl of 10% (w/v) SDS at room temperature overnight and their radiolabelled ligand content measured.

1. The use of a modified calycin monomer comprising:
i) a binding domain adapted to bind a ligand; and
ii) a targeting domain adapted to bind a substrate characterised in that the modified calycin monomer is used to target a ligand to a substrate, which is not hair or skin, and to which it would not naturally bind.

2. The use according to claim 1 wherein said modified calycin monomer is adapted to bind at least two ligands.

3. The use according to claim 2 wherein said ligands are the same.

4. The use according to claim 2 wherein said ligands are two different.

5. The use according to any of claims 1-4 wherein said modified calycin monomer is a multimeric complex.

6. The use according to claim 5 wherein said complex comprises identical calycin monomers.

7. The use according to claim 5 wherein said complex comprises different calycin monomers.

8. The use according any of claims 1-7 wherein said modified calycin monomer further comprises an interaction domain which facilitates the multimerisation of calycin monomers into a complex.

9. The use according to claim 8 invention said modified calycin monomers are multimerised by cross-linking agents.

10. The use according to claim 9 wherein said crosslinking agent is a bifunctional protein cross-linking agent.

11. The use according to claim 10 wherein said crosslinking agent is homo-bifunctional

12. The use according to claim 10 wherein said crosslinking agent is hetero-bifunctional.

13. The use according to claim 11 wherein said crosslinking agent is selected from the group consisting of: disuccinimidyl-suberimidate-dihydrochloride; dimethyladipimidate-dihydrochloride; 1,5-difluoro-2,4-dinitrobenzene.

14. The use according to claim 12 wherein said crosslinking agent is selected from the group consisting of: N-hydroxysuccinimidyl 2,3-dibromopropionate; 1-Ethyl3-[3-dimethylaminopropyl]carbodiimide hydrochloride; succinimidyl 4-[N-maleimidomethyl]-cyclohexane-1-carboxylate.

15. A modified calycin monomer which is altered by deletion, substitution or addition of at least one amino acid residue characterised in that said alteration alters the specificity of the binding domain for at least one ligand.

16. A modified calycin monomer according to claim 15 characterised in that said modification alters the affinity of said binding domain for at least one ligand.

17. A modified calycin monomer which is altered by deletion, substitution or addition of at least one amino acid residue characterised in that said alteration alters the specificity of the targeting domain for a selected surface.

18. A modified calycin monomer according to claim 17 chararcterised in that said modification alters the affinity of said targeting domain for a selected surface.

19. A modified calycin monomer according to any of claims 15-18 wherein said monomer is multimerised into a complex.

20. A vector which includes nucleic acid which encodes a modified calycin monomer according to any of claims 15-19.

21. A vector according to claim 20 wherein the vector is adapted for the recombinant production of said modified calycin monomer.

22. A cell transformed or transfected with the vector according claim 20 or 21.

23. A method to manufacture a modified calycin according to any of claims 15-19 comprising:
i) growing a cell according to claim 22 under conditions conducive to the manufacture of said calycin; and
ii) purifying said calycin from said cell, or its growth environment.

24. A method according to claim 23 wherein said vector encodes, and thus said calycin is provided with, a secretion signal or affinity tag to facilitate purification of the monomer.

25. The use according to any of claims 1-14 or a calycin according to any of claims 15-19 wherein said calycin is adapted for the targeting of at least one agent to the surface fibre of a laundry item.

26. The use according to claim 25 wherein said agent comprises a fragrance which confers a desired smell on said laundry item.

27. The use according to claim 25 or 26 wherein said agent comprises a conditioner which confers a desired texture to said laundry item.

28. The use according to any of claims 25-27 wherein said agent comprises a protective agent which confers a protective property to said laundry item.

29. The use according to any of claims 25-28 wherein said modified calycin is selected from the group consisting of: Mouse Urinary Protein (MUP) and equivalents thereof, rat alpha 2 microglobulin odour binding proteins (OBP), other odour-binding proteins and equivalents thereof, β-lactoglobulin, retinal and retinoid binding proteins, alpha 1 microglobulin, fatty acid binding proteins, intracellular retinoid binding proteins and pigmented lipocalins.

30. The use according to any of claims 1-14 or the calycin according to any of claims 15-19 wherein said calycin monomer or multimer is a biosensor.

31. The use according to claim 30 wherein said calycin is a pigmented lipocalin.

32. The use according to any of claims 1-14 or the calycin according to any of claims 15-19 for the targeting of at least one therapeutic agent to at least one surface.

33. The use according to claim 32 wherein the therapeutic agent is transferred into a cell to effect drug targeting, delivery and uptake.

34. The use according to claim 32 or claim 33 wherein said therapeutic agent is selected from the group consisting of: chemotherapeutic agents, antibiotics, neurological agents, receptor and ion channel modulators.

35. A method to multimerise modified calycin monomers comprising:
i) providing calycin monomers with conditions and crosslinking reagents sufficient to cross-link monomers into multimeric complexes; and optionally ii) purifying the cross-linked complexes from the reagent mix.
